(19) United States
(12) Patent Application Publication
(10) Pub. No.: US 2008/0260801 A1
(43) Pub. Date: Oct. 23, 2008

(54) COMPOSITE MATERIAL, ESPECIALLY FOR MEDICAL USE, AND METHOD FOR PRODUCING THE MATERIAL

(75) Inventors: Michael Ahlers, Eberbach (DE); Werner Badziong, Eberbach (DE); Christoph Gaissmaier, Tuebingen (DE); Juergen Fritz, Dusslingen (DE)

Correspondence Address: LEYDIG VOIT & MAYER, LTD, 700 THIRTEENTH ST. NW, SUITE 300, WASHINGTON, DC, 20005-3960, US

(73) Assignees: GELITA AG, Eberbach (DE); TETEC TISSUE ENGINEERING TECHNOLOGIES AG, Reutlingen (DE)

(21) Appl. No.: 12/120,366

(22) Filed: May 14, 2008

Related U.S. Application Data
(63) Continuation of application No. PCT/EP2006/010972, filed on Nov. 16, 2006.

(30) Foreign Application Priority Data
Nov. 17, 2005 (DE) 10 2005 054 940.3

(51) Int. Cl. A61K 9/00; A61K 35/12; A61P 43/00

(52) U.S. Cl. 424/426; 424/93.7

(57) ABSTRACT

A biocompatible, resorbable composite material having good mechanical properties, and can be populated by cells is provided comprising

a first self-supporting layer, which comprises a first material which is insoluble, resorbable and non-gelling under physiological conditions; and

a second layer, comprising a cross-linked, gelatinous second material, the second layer having a mainly open-pored structure.

### CROSS-REFERENCE TO RELATED APPLICATIONS

This patent application is a continuation of PCT Application No. PCT/EP2006/010972, filed Nov. 16, 2006, which claims priority of German patent Application No. 10 2005 054 940.3, filed Nov. 17, 2005, which are each incorporated by reference.

### BACKGROUND OF THE INVENTION

The present invention relates to a biocompatible, resorbable composite material, which is used in particular as a matrix material in the field of human and veterinary medicine. Materials of this kind may be used free of cells or also when populated with cells.

Further, the invention relates to a method for producing a composite material of this kind.

Finally, the invention relates to implants, in particular cell and tissue implants, which are produced using the composite material, and use of these implants for treatment of the human or animal body.

In the case of damage to many human or animal tissues, which may be caused both by illness and injury, resorbable implants are used to support the healing process. These promote regeneration of the tissue in question in that they perform a mechanical protective function for the newly forming tissue and/or provide a matrix which promotes cell growth.

An important field of use for implants of this kind is cartilage tissue. This consists of chondrocytes (cartilage cells) and the extracellular matrix synthesized by these cells, which is primarily built up from collagen and proteoglycanes. Since blood does not flow through cartilage, which is predominantly nourished by diffusion and has no direct access to regenerative cell populations when epiphyseal fusion has terminated, cartilage has only limited capability for intrinsic regeneration. Stand-along healing of cartilage damage is therefore only possible to a very limited extent, above all in the case of adults, and is rarely observed. Cartilage defects may occur due to injuries or degenerative effects, and without biologically reconstructive intervention, often lead to further advance of the cartilage damage right up to destructive osteoarthritis.

In the case of a specific form of treatment for cartilage damage as described above, chondrocytes are first of all cultivated in vitro on a resorbable implant using a nutrient solution. The cell-carrier construct produced in this way in then inserted in place of the missing or damaged cartilage. The cultivated chondrocytes are previously taken from the patient himself, so that this method may also be referred to as transplantation of autologous cartilage cells. After implantation, the cells produce a new extracellular matrix and thus lead to healing of the defect. The carrier material is broken down (resorbed) in the course of the regeneration. Apart from the use of autologous chondrocytes, implantation of allogenic chondrocytes or use of stem cells which have been pre-differentiated chondrogenically (autologously or allogenically) in vitro is also conceivable, and is at present being evaluated in preclinical and experimental research on animals for clinical usability in humans.

Along with autologous chondrocyte transplantation, bone-marrow-stimulating methods, such as microfracture or boring-in, provide a further clinically established therapy having a biologically reconstructive purpose in the case of cartilage damage. In these methods, the subchondral bone plate is perforated with small awls or drills, after previous debridement, by virtue of which blood flow takes place into the region of the defect with a blood clot being formed. In the further course of events, a fiber cartilage develops from the blood clot (a so-called superclot), which in many cases leads to filling up of the defect and alleviation of the problem. The results of this method may be further improved by the use of suitable and biocompatible matrices. The biomaterial used fixes, in the region of the defect, the superclot which has developed, protects it from shear, and acts as a primary matrix for the cells which migrate by of the blood path, for healing of the defect.

A further field of use for biomaterials is in the treatment of ruptures of the rotator cuff of the shoulder or the treatment of partial degeneration of the rotator cuff. While cell-free biomaterials for these indications are already known, they have however the disadvantage that without prior population with cells they cannot contribute actively to regeneration. For vitalizing the material, seed tissue may be taken by biopsy. The cells may then be isolated in vitro, cultivated, seeded-out onto a suitable biomaterial and implanted, along with the biomaterial, into the region of the defect.

A further use for a cell-populated biomaterial is bone regeneration, for example in the jaw region for sinus augmentation, using pre-cultivated autologous cells of the periosteum or mesenchymalic stem cells, which are seeded-out onto the matrix.

As well as the indications mentioned so far, biomaterials may also be used in connection with or without prior cell population for treatment and healing of chronic wounds, skin injuries or bums of the skin.

In order for biomaterials suitable for the indications and methods described above to be usable for humans or animals, a series of requirements must however be met. Of great importance among these is first of all complete biocompatibility of the material, i.e. there should be no inflammation reactions, rejection reactions or other immune reactions after implantation. In addition, the biomaterial should exercise no negative effect on the growth or the metabolism of the transplanted or migrating cells and should be completely resorbed in the body after a specific time. Moreover, the material should have a structure such that it is populated and penetrated by cells as uniformly as possible.

At the same time, high demands are also to be placed on the mechanical properties of the material used. Safe handling of the material during implantation, without its being damaged, is only to be assured by high mechanical strength. In particular, this strength must also be provided for tissue implants which have already been populated with cells.

Recent developments show that these demands are most likely to be met by multi-layer composite materials. For example, a multilayer membrane is described in WO 99/19005 which comprises a matrix layer of type II collagen with a sponge-like texture and at least one barrier layer with a closed, relatively impermeable texture.

In EP 1 263 485 B1, a biocompatible multilayer material is disclosed, which has a first and a second layer with matrices of biocompatible collagen.

Collagen is a natural material with relatively high strength, on the basis of which implants with good mechanical properties and good ability to be handled may be produced. On the other hand, use of collagen as a matrix for cells has however the disadvantage that on account of the less than precisely reproducible composition and purity of collagen, problems may occur in respect of biocompatibility. Furthermore, the resorption time of materials containing collagen is not very controllable, but control of resorption time would be desirable for the various fields of use.

### SHORT SUMMARY OF THE INVENTION

It is an object of the present invention to make available a composite material in which these disadvantages are avoided as far as possible, and that has improved properties compared with known materials.

This object is met according to the invention in the case of composite material of the kind mentioned at the beginning by the composite material comprising the following two layers:
a first self-supporting layer, which comprises a first material which is insoluble, resorbable and non-gelling under physiological conditions; and
a second layer, produced based on a cross-linked, gelatinous second material, the second layer having a mainly open-pored structure.

In the case of the composite material according to the invention, the first layer ensures the required mechanical strength while the second layer forms a matrix for growth of cells.

### DETAILED DESCRIPTION OF THE INVENTION

The first material is insoluble and non-gelling under physiological conditions. In the sense of the present invention, this means that the material is not physically dissolved in an aqueous solution under the conditions prevailing in the body (in particular temperature, pH value and ion strength) and also is not transformed into a gel or a gel-like state by take-up of water. Gel formation in this sense is therefore present when the first material loses thereby its original strength and shape-retaining ability to a substantial extent. This does not exclude the material taking up certain quantities of water and thereby possibly also swelling up, as long as this does not lead to any significant impairment of the mechanical strength.

By virtue of the properties quoted, the first material also remains mechanically firm and stable as to shape, even in a hydrated state, whereby the first layer is given its self-supporting function. This means that not only can the first layer be handled without any additional carrier, but that it is, for its part, in a position to serve as carrier for the second layer.

At the same time, the first material is resorbable, i.e. it is broken down by hydrolysis after a specific time in the body. Enzymes may also play a part in this hydrolytic degradation. Before resorption in the body takes place, thus in particular during the cultivation of cells on the composite material in vitro and during implantation of the composite material, the carrier function of the first layer is largely unimpaired, whereby the composite material as a whole is given the required mechanical strength.

By virtue of the embodiment, according to the invention, of the first layer, safe and damage-free handling of the composite material is assured. This also applies in particular in the case when the second layer is already populated with cells before implantation.

Furthermore, the first layer also offers mechanical protection for the cells after the composite material has been implanted. This is meaningful both for transplantation of cells precultivated in vitro as well as for microfracturing linked with a matrix. For both methods, the biomaterial is advantageously used in such a way that the first layer is oriented outwardly away from the bone. This then protects the growing cells in the second layer from shear and from regeneration-disturbing influences from the interior of the joint, such as for example excessive mechanical load.

A further advantage which touches on the high strength of the first layer is the surgical stitchability or also the exercise-stable subchondral fixing of the composite material according to the invention, by means of a resorbable fixing. The first layer preferably has a tear strength such that the composite material does not tear when it is stitched or when it undergoes transossar fixation by means of resorbable minipins.

Preferably the first layer has a tear strength of 20 N/mm² or more.

In a preferred embodiment of the invention, the insoluble, resorbable and non-gelling first material is a planar material based on collagen. Among such materials are planar materials which are formed substantially from collagen and for which preferably natural membranes of animal origin are in question. Animal membranes, which consist almost entirely of collagen, may be obtained, the membranes being made free of foreign constituents which would have disadvantageous effects on biocompatibility.

Animal membranes provide as a rule high strengths and are therefore especially well-suited for the first layer of the composite material according to the invention. In particular, collagen exhibits the required properties to the extent that it is, under physiological conditions, insoluble, non-gelling and resorbable.

As a preferred planar material based on collagen, a pericardial membrane is used as first layer of the composite material. The pericardium is the outer layer of the heart sac, this representing a particularly tear-resistant animal membrane. For example, the pericardial membrane of cattle may be used.

The pericardial membrane has, as do many other animal membranes, a rough side and a smooth side. Preferably such membranes are used in the composite material in such a way that the rough side is oriented toward the second layer. The stability of the bond between the two layers is increased because of the roughness of the surface.

In a further preferred embodiment of the composite material according to the invention, the first material comprises a reinforcing material. The strength of the first layer can also be increased by means of insoluble, resorbable, non-gelling reinforcing materials to such extent that it has the advantageous properties described above.

When reinforcing materials are used as first material, the first layer preferably comprises a matrix into which the reinforcing material is embedded. The first layer is then for example a reinforced film. The matrix must for this likewise be resorbable and comprise preferably gelatin.

A gelatin-comprising matrix for the first layer, for example, a gelatin film, is preferably produced based on a cross-linked, gelatinous material. Cross-linking is as a rule required in order to convert the material into an insoluble form. Preferred embodiments for the cross-linking of the gelatinous material, in particular gelatin itself, are explained further below in connection with the second layer of the composite material.

The reinforcing material shows, even at fractions of 5% by weight with reference to the mass of the first layer, a marked improvement in the mechanical properties of the layer.

Above 60% by weight, no further significant improvement can be achieved and/or the desired resorption properties or also the necessary flexibility of the first layer can be achieved only with difficulty.

The reinforcing material may be selected from particulate and molecular reinforcing materials as well as mixtures of these.

In regard to particulate reinforcing materials, the use of reinforcing fibers is in particular to be recommended. For this, the fibers are preferably selected from polysaccharide fibers and protein fibers, in particular collagen fibers, silk and cotton fibers, and from polyactide fibers and mixtures of any of the foregoing.

On the other hand, molecular reinforcing materials are likewise suitable in order to improve the mechanical properties and, if desired, also the resorption stability of the first layer.

Preferred molecular reinforcing materials are in particular polyactide polymers and their derivatives, cellulose derivatives, and chitosan and its derivatives. The molecular reinforcing materials may also be used as mixtures.

The second layer of the composite material according to the invention is that layer which comes directly into contact with the cells in medical use and should therefore be in a position to function as a substrate for population with cells and as a matrix for their growth. For this reason, especially high demands are placed on the biocompatibility (i.e. cell compatibility) of the second material. Since the first layer already ensures the required mechanical strength of the composite material and fulfils a support function for the second layer, the selection of material and structure for the second layer can be determined wholly on its biocompatibility and biological functionality.

The above-mentioned requirements for the second layer are fulfilled to a great extent by use, according to the invention, of gelatin. Gelatin is, in contrast to collagen, obtainable with a defined and reproducible composition as well as with high purity. It has excellent tissue and cell compatibility and is resorbable to leave no residue.

Preferably, the second material is formed to a predominant extent from gelatin, more preferably it is formed substantially entirely from gelatin.

In order to ensure optimal biocompatibility of the second layer of the composite material according to the invention in medical use, the second material preferably comprises a gelatin with a particularly low content of endotoxins. Endotoxins are metabolic products or fragments of microorganisms, which are present in animal raw material. The endotoxin content of gelatin is specified in International Units per gram (I.U./g) and is determined by the LAL test, the carrying out of which is described in the fourth edition of the European Pharmacopoeia (Ph. Eur. 4).

In order to keep the content of endotoxins as low as possible, it is advantageous for the microorganisms to be killed off as early as possible in the course of preparation of the gelatin. Furthermore, suitable standards of hygiene are to be observed in the preparation process.

Accordingly, the endotoxin content of the gelatin can be drastically reduced during the preparation process by specific measures. Among these measures, there belong primarily use of fresh raw materials (for example, pig skin) with storage time being avoided, meticulous cleaning of the entire production installation immediately before beginning preparation of the gelatin, and optionally replacement of ion exchangers and filter systems in the production installation.

The gelatin used within the scope of the present invention preferably has an endotoxin content of 1,200 I.U./g or less, still more preferably, 200 I.U./g or less. Optimally, the endotoxin content is 50 I.U./g or less, in each case determined in accordance with the LAL test. By comparison with this, many commercially available gelatins have endotoxin contents of more than 20,000 I.U./g.

According to the invention, the second gelatinous material is cross-linked, the gelatin preferably being cross-linked. Since gelatin is in itself water-soluble, cross-linking is as a rule required, in order to prevent unduly speedy dissolving of the second material, and thereby also ensure a sufficient lifespan for the second layer of the composite material under physiological conditions.

Gelatin then offers the further advantage that the speed of resorption of the cross-linked material, or the time period up to complete resorption, may be set over a wide range by choice of the degree of cross-linking.

The second material is preferably cross-linked chemically. In principle, all compounds may be used as cross-linking agents which effect chemical cross-linking of gelatin. Preferred are aldehydes, dialdehydes, isocyanates, diisocyanates, carbodiimides and alkyl halides. Particularly preferred is formaldehyde, since this also has a sterilizing effect.

In order to ensure the biocompatibility of the second material, this is preferably substantially free from excess cross-linking agent, i.e. cross-linking agent which has not reacted. Preferably for this the content of excess cross-linking agent is about 0.2% by weight or less, this in particular in the case of formaldehyde representing a limiting value for its allowability as an implant material.

In a further embodiment, the second material is cross-linked enzymatically. For this, the enzyme transglutaminase is preferably used as cross-linking agent, this effecting linking of glutamine and lysine side chains of proteins, in particular also of gelatin.

The cross-linking agents specified are likewise suitable for cross-linking the gelatinous material of the first layer, in the case where this comprises a gelatinous matrix with an embedded reinforcing material.

As well as biocompatibility of the material used, the second layer of the composite material should also be created in such a way that it has a structure suitable for population with cells. According to the invention, this is assured by the mainly open-pored structure, which enables penetration of cells into the structure as well as the most uniform possible distribution of cells over the entire thickness of the second layer.

The mainly open-pored structure is realised, in a preferred embodiment of the invention, by the second layer having a fiber structure. The fiber structure comprises preferably a textile, a knitted material, or a non-woven material. Fiber structures may be produced from the gelatinous second material, for example by extrusion or electrospinning of a gelatin solution.

In a further preferred embodiment of the composite material according to the invention, the second layer has a sponge structure. Sponge structures can be produced by foaming a solution of the gelatinous second material, which will be gone into in more detail in connection with the method of production according to the invention.

Sponge structures with mainly open pores are especially suitable for population with cells. By virtue of the hollow spaces being connected with one another, very uniform distribution of the cells may be achieved over the entire volume. A three-dimensional tissue structure is thus formed during growth of the cells and synthesis of the extracellular matrix. This is accompanied by successive hydrolytic breakdown of the cross-linked, gelatinous material, so that the volume of the sponge structure, after complete degradation of the material (or after its resorption in the body), is taken up to a great extent by the newly-formed tissue.

The preferred average pore diameter of the sponge structure is matched primarily to the size of the cells with which the composite material is to be populated in vitro or in vivo. If the pore diameters are too small, the cells cannot penetrate into the structure, whereas if the pores are too large, the result is too little support when the cells are introduced or grown in. Preferably, the average pore diameter is below 500 μm, in particular in the range from 100 to 300 μm.

The pore size of the sponge structures is to a great extent dependent on their density. The density of the second layer of the composite material, in particular in the case of a sponge structure, is preferably in the range from 10 to 100 g/l, more preferably 10 to 50 g/l, most preferably 15 to 30 g/l. The density of sponge structures may for this be influenced by production conditions, in particular by the intensity of foaming.

Preferably, the second layer of the composite material according to the invention is elastically deformable in a hydrated state, in particular in the case of a sponge structure. A hydrated state exists when the composite material in an aqueous environment has taken up so much water that an equilibrium state is substantially reached. Conditions of this kind are present both in the case of cultivation of cells in a nutrient medium in vitro and also in the body.

A measure of elastic deformability may be defined for example by the decompression behavior. Preferably the second layer is formed so that after it has undergone a compression in volume by action of a pressure of 22 N/mm², in a hydrated state, it decompresses to 90% or more within 10 minutes, this not being achievable as a rule with material based on collagen. In order to measure the decompression ratio in a hydrated state, the material to be tested is put into PBS buffer (pH 7.2) at 37° C.

Elastically deformable structures of this kind lead to flexibility of the second layer of the composite material which is extremely advantageous for use of the material as an implant. The composite material can therefore be well adapted to the shape of the tissue defect to be treated, which is frequently irregular or at least curved, as for example in the case of damage to joint cartilage.

A further advantage of the composite material according to the invention is that the second layer, in the hydrated state, exhibits no significant diminution in volume. In particular in the treatment of cartilage defects, where the precisely fitting pieces of composite material are inserted into the surrounding cartilage, shrinkage of this kind, such as is observed in the case of porous materials based on collagen, leads to significant problems. Preferably, the second layer, after three days in a hydrated condition, has a reduction in volume of less than 5% compared with the volume measured after 5 minutes. It is most advantageous if the volume of the second layer is slightly increased in the hydrated state.

As already stated, the composite material according to the invention offers the particular advantage that the speed of resorption of the second layer may be adapted to individual requirements. This can in particular be effected by selection of the density of the second layer and the degree of cross-linking of the gelatinous, second material, both higher density and a higher degree of cross-linking leading to a tendency toward prolongation of lifespan. In the ideal case, the breakdown of the material is effected in accordance with the extent to which the extracellular matrix is synthesized from the cells. This can be very different according to the type of cell, cartilage cells in particular having comparatively slow growth and therefore involving a tendency toward longer breakdown times for the second layer.

A measure for the speed of resorption or degradation of the second layer when populated with cells may also be derived from its stability without cell population under standard physiological conditions (PBS buffer, pH 7.2, 37° C.). The physiological conditions to which the composite material is exposed, are distinguished primarily by temperature, pH value and ion strength, and may be simulated by incubation of the composite material under the standard conditions mentioned, in order to test and compare different materials in respect of their time-dependent breakdown behavior.

According to the invention, composite materials may be obtained by changing the production conditions, for which, under standard physiological conditions, the second layer remains stable for example for longer than a week, longer than two weeks and longer than four weeks

The concept of stability is to be understood as the second layer substantially retaining its original shape (macroscopic geometry) during the respective time period and only then degrading to an extent visible from the outside.

In the case where the second layer has a sponge structure, this degradation takes place relatively suddenly after the respective time period, the sponge structure disintegrating within a few days.

Alternatively, the breakdown behavior of the second layer may also be defined by the loss of weight under the conditions described above. Accordingly, composite materials according to the invention may be obtained in which the second layer is still comprised of to 70% or more by weight after one week, after two weeks or after four weeks.

A further advantage of the structure of the second layer is that it can be converted into a hydrogel-like state during the resorption phase. Conversion of this kind into a hydrogel-like structure under standard physiological conditions is in particular of advantage for stabilising phenotypes of chondrogenic cells. These properties support tissue reconstruction of a high qualitative value compared with other biomaterials. On the other hand, biomaterials which are primarily gel-like allow a clearly worse cell population and hardly any cell growth (for example after microfracture) in their relatively closed structures.

The convertibility of the structure of the second layer into a hydrogel-structure is then dependent on the degree of cross-linking. It does not contradict the above mentioned stability, since this relates to the macroscopic geometry of the second layer, which initially remains extant even in the presence of the hydrogel structure.

The degradation time for the first layer of the composite material according to the invention may deviate from that for the second layer and may be chosen to be longer or shorter, depending on the circumstances. In every case however, the first layer based on the first material according to the invention provides a sufficient lifespan to ensure that the first layer has its self-supporting property even after cultivation of cells in the second layer and gives to the composite material, the mechanical strength required for implanting.

If for example a reinforced gelatin is used as the first layer, its degradation time may be set in a specific range by way of the degree of cross-linking of the gelatin, as in the case of the gelatinous material of the second layer. When a membrane of animal origin is used, its breakdown time is largely predetermined and is in most cases greater than that of the second layer.

The first and second layers of the composite material according to the invention are preferably bonded directly to one another. This may for example be achieved by the second layer being prepared directly on a surface of the first layer, in particular on the rough side of a animal membrane.

In another embodiment of the composite material according to the invention, the two layers are bonded to one another by means of an adhesive, the adhesive preferably comprising gelatin.

The composite material according to the invention preferably has a thickness of 2 to 5 mm, a thickness of up to 3 mm being further preferred. The thickness of the first layer is then preferably about 1 mm or less.

The thickness of the composite material mentioned relates therefore to the total thickness of the first and the second layer. The composite material according to the invention may however furthermore comprise still more layers.

In a particular embodiment, a third layer is provided which is bonded to the second layer, this third layer being produced based on a gelatinous material. A third layer of this kind serves, for example in the case of transplantation of cells pre-cultivated in vitro, to protect cells located in the second layer from mechanical load or from the growth of foreign cells, or to improve the bonding of the composite material to the neighbouring tissue during implanting.

In order to fix an implant at its prescribed position in the body, in particular to a bone in the case of cartilage cell transplantation, a gelatin solution may be used for example as third layer, the gelatin solution being applied as adhesive to the second layer.

The gelatinous material of the third layer is preferably cross-linked, in particular the gelatin itself. Preferred cross-linking agents for this are the compositions and enzymes described in connection with the second material of the second layer.

The third layer advantageously has a structure which prevents or impedes the penetration of foreign cells, for example bone cells in the case of cartilage transplantation. The third layer preferably has therefore a substantially closed structure. By this there is meant a structure without pores or passages, in particular a film, for example, a gelatin film.

Alternatively the third layer may also have a porous structure, the average pore diameter of which is less than the average pore diameter of the structure of the second layer. There is therefore in question a sponge structure as described in connection with the second layer, the sponge structure of the third layer preferably having an average pore diameter of 300 μm or less, in particular 100 μm or less. The third layer preferably also has a higher density than the second layer, preferably a density of 50 g/l or more.

By virtue of a third layer with a closed or porous structure, the bond between the composite material and the neighbouring tissue, especially bone, may also be improved. The degree of cross-linking of the material of the third layer is therefore selected to be relatively low, so that the material partially gels and thus functions as adhesive.

For use of the composite material in transplantation of pre-cultivated cells, such as for example cartilage cells or mesenchymalic stem cells, the third layer may be optimised in respect of good compatibility with bone. Preferably, the third layer then comprises one or more calcium phosphates, apatites, or mixtures thereof.

The third layer of the composite material is preferably applied to the second layer after cells have been introduced into and cultivated in the second layer. Alternatively, cells may be introduced into the second layer from the side after the third layer has been applied, this being easily possible in the production of smaller implants.

The present invention has further the object of providing a method for producing above-described composite material.

This object is met according to the invention in the case of the method mentioned at the beginning by the method comprising:
providing a first self-supporting layer, which comprises a first material which is insoluble, resorbable and non-gelling under physiological conditions;
production of a second layer based on a cross-linked, gelatinous second material, so that the second layer has a mainly open-pored structure; and
bonding the first and the second layer, the composite material being formed.

The bonding of the two layers may according to the invention be effected as the final method step or in the course of preparation of the second layer.

In first instance, the bonding is preferably by means of an adhesive. For this, the adhesive preferably comprises gelatin, which for example may be applied in the form of a solution to one or both layers, after which the layers are joined together and dried.

In the case where the first layer comprises a gelatinous matrix, it is further preferred for the prepared second layer to be pressed partially into the first layer. This can for example be effected by the gelatinous matrix, for example a gelatin film, being in a plastically deformable condition during the pressing-in of the second layer, for example in a wettish condition after preparation of the matrix.

A preferred embodiment of the method according to the invention relates to composite materials, in which the second layer has a sponge structure. The bonding of the two layers is effected in the course of producing the second layer, the method comprising the following steps:

a) providing the first layer;
b) preparation of an aqueous solution of the gelatinous second material;
c) partial cross-linking of the dissolved second material;
d) foaming of the solution;
e) application of the foamed solution to the first layer; and
f) leaving the foamed solution to dry, the second layer being formed to have a mainly open-pored structure.

For this method, basically gelatin of diverse origin and quality may be used as starting material; in regard to medical use of the composite material, gelatin which is low in endotoxins, as described above, is however preferred. The solution in step b) preferably has a gelatin concentration of 5 to 25% by weight, in particular 10 to 20% by weight.

Apart from gelatin, the second material in the method according to the invention may contain still further constituents, for example other biopolymers.

For the cross-linking reaction in step c), one, several or all constituents of the dissolved second material may in this case be partially cross-linked. Preferably in this, the gelatin in particular is cross-linked. Cross-linking may be effected chemically or enzymatically, preferred cross-linking agents having been already described in connection with the composite material according to the invention.

Another preferred embodiment of this method comprises a further step g) in which the second material comprised in the second layer is in addition cross-linked.

The advantage of two-stage cross-linking of this kind is that a higher degree of cross-linking of the second material can be achieved and thereby as a result the advantageous longer resorption times for the second layer. This cannot be realised to the same extent with a single-step method by increasing the concentration of cross-linking agent, because if the cross-linking of the dissolved material is too strong, this can no longer be foamed and shaped.

On the other hand, cross-linking of the material, in particular the gelatin, exclusively after preparation of the composite material is not suitable, because the material is thereby more strongly cross-linked at the delimiting surface accessible from the outside than in the inner regions, this being reflected in non-homogeneous breakdown behavior.

The second cross-linking (step g)) may be carried out by the action of an aqueous solution of a cross-linking agent, for which the above-described chemical or enzymatical cross-linking agent may be used. Preferred however is the action of a gaseous cross-linking agent, in particular formaldehyde, which at the same time has a sterilizing effect. The action of the formaldehyde can for this be effected on the composite material, facilitated by a steam atmosphere.

The cross-linking agent in step c) is preferably added to the solution in an amount of 600 to 5,000 ppm, preferably 1,000 to 2,000 ppm, with reference to the gelatin.

By variation of the concentration of cross-linking agent in the solution, but also by differently high degrees of cross-linking in the second cross-linking step, the lifespan of the second layer of the composite material may be easily set. Surprisingly, sponge structures can be obtained which, under physiological conditions, remain stable for example for longer than one week, longer than two weeks, or longer than four weeks, as has been already explained in detail in connection with the composite material according to the invention.

The foaming, (step d)), is effected preferably by introducing a gas, in particular air, into the solution. The density and the average pore diameter of the sponge structure to be produced may thereby be adjusted over a wide range, preferably by means of the intensity of foaming. Apart from matching the average pore diameter to the cells with which the second layer is to be populated, the flexibility and elastic deformability of the second layer may also be influenced by these parameters (and thereby the flexibility and elastic deformability of the composite material as a whole). High flexibility is for example desirable in order to be able to match, in an optimal manner, an implant to the shape of the tissue defect to be treated.

The properties of the composite material produced in accordance with this method may be still further improved in respect of the stability of the second layer if the composite material is exposed to a thermal after-treatment at reduced pressure, after the second cross-linking. This thermal after-treatment is preferably carried out at temperatures from 80 to 160° C., since below 80° C., the observed effects develop to only a relatively weak extent, while above 160° C., an unwanted coloration of the gelatin may occur. Mostly, values in the range from 90 to 120° C. are preferred.

At reduced pressure is to be understood here as pressures of less than atmospheric pressure, the lowest possible pressure values, in the ideal case a vacuum, being preferred.

The thermal after-treatment acts advantageously in two aspects. On the one hand, the above-mentioned temperature and pressure conditions effect a further, dehydrothermal cross-linking of the gelatin, in that different amino acid chains react with each other with the elimination of water. This is favoured by the water eliminated being taken out of the equation by the low pressure. By virtue of the thermal after-treatment, a higher degree of cross-linking can therefore be achieved for the same quantity of cross-linking agents, or the quantity of cross-liking agents can be reduced for a comparable degree of cross-linking.

The further advantage of the thermal after-treatment resides in the residue of unused cross-linking agent remaining in the second layer being markedly reduced.

In order to ensure good biocompatibility of the composite material, excess cross-linking agent, which has not reacted, is preferably removed from the second layer, in the method according to the invention. This may for example be effected by degassing the composite material for several days at normal pressure and/or by washing with a fluid medium, the latter requiring likewise a time period from one day to a week depending on the concentration of the cross-linking agent, the size of the composite material and so on.

Since by the above-described thermal after-treatment, on the one hand, the quantity of cross-linking agent used can be reduced and moreover, excess cross-linking agent can be removed from the composite material by virtue of the raised temperature and the reduced pressure, a marked reduction in the residue of cross-linking agent can be achieved by this additional method step, even within about 4 to 10 hours.

In a particular embodiment of the method according to the invention, this comprises further application of a third layer to the second layer of the composite material. This may take place both before introduction of cells into the second layer or after this. Advantages and embodiments of a third layer have already been described in connection with the composite material according to the invention.

The invention further relates to usage of the composite material described for use in the fields of human and veterinary medicine, in particular for producing implants.

The composite material according to the invention is exceedingly suitable for population with human or animal cells, or for the growth of such cells. For transplantation of cells which have been isolated and/or pre-cultivated in vitro, the composite material is populated for example with chondrocytes, mesenchymalic stem cells, periosteum cells or fibroblasts, which are seeded-out onto the second layer in a suitable nutrient medium and preferably embedded into the mainly open-pored structure of this layer. Because of the high stability of the material, the cells can grow and proliferate in vitro for several weeks.

The invention relates furthermore to implants, in particular tissue implants, which comprise the composite material and human or animal cells.

In one embodiment of the implant according to the invention, this comprises only growing cells, which are embedded in the second layer. In this case, loading of the cells in vitro does not take place, but the composite material is implanted directly, for example after previous microfracture. The cells in the blood clot then populate the biomaterial in vivo.

In a further embodiment of the implant according to the invention, the cells are cultivated in the second layer, i.e. population and cultivation is carried out in vitro before implantation, as described above.

The cells growing in vivo and/or seeded-in in vitro are preferably substantially uniformly distributed in the second layer of the composite material. In this way, the formation of a three-dimensional tissue structure is made possible.

The implants according to the invention are used for treatment of tissue defects, as have already been discussed several times. Preferred uses relate to treatment of damage and/or injuries of human or animal cartilage, in particular in the context of autologous cartilage cell transplantation or matrix-linked microfracture, treatment of defects in the rotator cuff of the shoulder, bone defects (for example sinus augmentation of the jaw), as well as treatment of damage, injuries and/or bums of the human or animal skin.

Here also, the composite material according to the invention facilitates a protected and direct rehabilitation of defects in the sense of guided tissue regeneration, on account of its structure.

Finally, the invention relates to, as already mentioned, a method for cell-based cartilage regeneration with cells cultivated in vitro. The method comprises taking chondrocytes or stem cells of autologous or allogenic origin, seeding-out potentially chondrogenic cells onto the second layer of a composite material according to the invention, and the insertion of the composite material with the cells at the location of the cartilage defect in a patient.

The shape of the composite material is for this preferably matched to the shape of the cartilage defect. Further, it is preferred for the first layer of the composite material to be oriented outwardly when it is inserted into the cartilage.

In a preferred embodiment of the method, the seeded-out cells are cultivated in vitro before implantation of the composite material, preferably for a time period of 4 to 14 days.

### SHORT DESCRIPTION OF THE SEVERAL VIEWS OF THE DRAWINGS

These and further advantages of the invention will be explained in more detail on the basis of the accompanying examples with reference to the figures. In particular:

FIG. 1: shows an image, taken using an optical microscope, of a cross-section through a composite material according to the invention;

FIG. 2: shows an image, taken using an optical microscope, of the second layer of a composite material according to the invention after a two-week period of population with chondrocytes; and

FIG. 3: shows a photographic illustration of a composite material according to the invention after a four-week period of population with chondrocytes.

### EXAMPLES

### Example 1

### Production and Properties of a Composite Material According to the Invention

This example relates to the production of a composite material according to the invention, in which a pericardial membrane from cattle is used as first layer.

In order to guarantee the highest possible biocompatibility, a pericardial membrane was used that had be made free of fats, enzymes and other proteins to the greatest possible extent. A loose fiber structure for the collagen was obtained by lyophilisation of the membrane. Pericardial membranes of this kind, which consist substantially of type I collagen, are also used to replace connective tissue structures in neurosurgery.

Three pieces of this pericardial membrane, each about 10×10 cm² in size, were fixed, with the rough side upward, onto underlay blocks about 3 cm high. These three blocks were then distributed on the floor of a box mold having a length and breadth of 40×20 cm² and a height of 6 cm.

In order to produce the second layer of the composite material, first of all a 12% by weight solution of pig skin gelatin with a Bloom strength of 300 g was prepared, the gelatin being dissolved in water at 60° C. The solution was degassed by means of ultrasound and an appropriate quantity of an aqueous formaldehyde solution (1.0% by weight, room temperature) was added, so that 2,000 ppm of formaldehyde were present, relative to the gelatin.

The homogenized mixture was brought up to 45° C. and after a reaction time of 5 minutes, it was mechanically foamed with air for a period of about 30 minutes, a gelatin foam with a wet density of 130 g/l being obtained.

The box mold with the tensioned pericardial membranes was filled up with this foamed gelatin solution, which had a temperature of 27° C., and the gelatin foam was dried for about 6 to 8 days at a temperature of 26° C. and a relative humidity of 10%.

After drying, the gelatin foam formed a firm material with a mainly open-pored sponge structure (called gelatin sponge in the following text). By drying the gelatin foam in direct contact with the pericardial membrane, there resulted a stable bond between the two materials over the greater part of their areas, this being in addition promoted by the roughness of the surface used on the pericardial membrane.

Pieces of the pericardial membrane about 1.5×1.5 cm² in size, together with the gelatin sponge adhering to it, were cut off, the gelatine sponge above the membrane being cut away to the extent that the pieces had a thickness of about 3 mm.

The gelatin sponge forming the second layer of the composite material has, in the foregoing example, after drying, a density of 22 g/l and an average pore diameter of about 250 μm. By changing the production circumstances, these parameters may be controlled over a broad range in order to match the average pore diameter to the size of the cells by which the composite material is to be populated.

Thus by changing the intensity of the foaming for example, composite materials may also be produced in accordance with the procedure described above in which the gelatin sponge has a wet density of 175 g/l, a dry density of 27 g/l and an average pore diameter of about 200 μm, or a wet density of 300 g/l, a dry density of 50 g/l and an average pore diameter of about 125 μm.

In order to ensure a sufficiently lengthy lifespan for the second layer of the composite material, the gelatin was submitted to a second cross-linking step. For this, pieces of the carrier material, each 1.5×1.5 cm² in size were exposed, in a dessicator, for 17 hours to the equilibrium vapor pressure of an aqueous formaldehyde solution of 17% by weight, at room temperature, the dessicator having been previously evacuated two or three times and recharged with air.

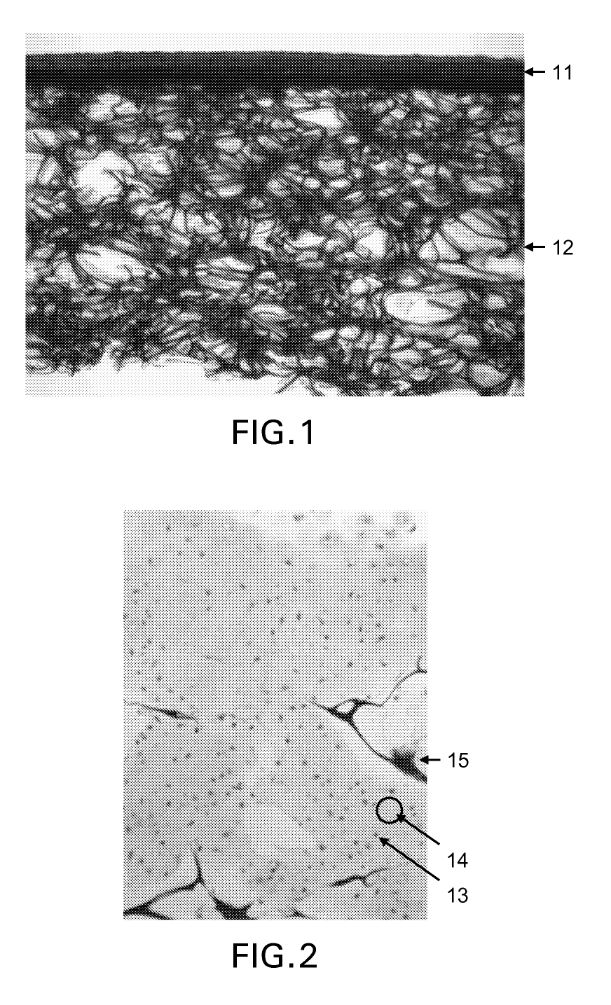

In FIG. 1, there is illustrated an image taken with an optical microscope of a cross-section through the composite material according to the invention produced in this way. In this, the first layer is formed by the pericardial membrane 11 and the second layer 12 is formed by the gelatin sponge with the average pore diameter of about 250 μm. The predominantly open-pored structure of the second layer is clearly to be seen.

In order to demonstrate the effect of the second cross-linking step, the breakdown behavior of composite material which had been cross-linked twice was compared with that of composite material which had been cross-linked once. For this, test pieces of the composite material described above, each about 1.5×1.5 cm² in size, as well as reference samples which had not been exposed to any subsequent cross-linking in the gas phase, were placed in 75 ml PBS buffer (pH 7.2) and stored at 37° C.

This showed that in the case of the samples of the composite material with gelatin that had been cross-linked only once, the second layer was fully broken down after only three days. By contrast, for the samples which had been exposed to the subsequent cross-linking in the gas phase, described above, the second layer was still extant to the extent of more than 80by weight, even after 14 days. For all samples, there was still no degradation to be seen at the pericardial membrane of the first layer, after 14 days.

It must in this connection naturally be noted that in the case of population of the composite material with cells or when it is in the body, the actual times for breakdown may differ from the times found in this experiment. Nonetheless, this result shows that the lifespan of the second layer under physiological conditions can be markedly prolonged by two-stage cross-linking of the gelatin, which is of significant importance for medical use of the composite material, in particular in the field of cartilage transplantation.

Moreover, it is possible to influence the lifespan in a targeted manner by variation of the production conditions. In particular, a higher fraction of cross-linking agent in the gelatin solution, a higher density of the gelatin sponge and/or a longer time of exposure to the cross-linking agent in the gas phase, lead to prolongation of the breakdown times.

In addition, the lifespan may also be prolonged further by a thermal after-treatment. This may in the present example take place by the sample pieces being degassed by vacuum after the second cross-linking step and then being held under a vacuum of about 14 mbar by means of a rotational evaporator for six seconds at 105° C.

If a thermal after-treatment of this kind is carried out, the reaction time of 17 hours for the formaldehyde in the second cross-linking step may be shortened to for example two or five hours, in order to achieve a composite material with a lifespan for the second layer in the range from one to four weeks. By virtue of this procedure, the second layer also has a residue of excess formaldehyde which is reduced by up to 40%. The time for which the composite material according to the invention requires to be washed, before it is implanted or populated with cells, is thereby shortened.

### Example 2

### Production of Another Composite Material According to the Invention

This example relates to the production of a composite material according to the invention, in which a gelatin film reinforced with cotton fibers is used as first layer.

In order to produce the first layer, 20 g of pig skin gelatin (Bloom strength 300 g) was dissolved at 60° C. in a mixture of 71 g of water and 9 g of glycerin and the solution was degassed by means of ultrasound. The glycerin served in this as a plasticizer, in order to ensure a certain flexibility and stretchability of the gelatin film produced.

1 g of short cotton fibers (linters) were formed into a slurry in 25 g of water, as reinforcing material, and this suspension was added with continual stirring to the solution of gelatin and glycerin. After addition of 2 g of an aqueous formaldehyde solution (2.0% by weight, room temperature) to the solution, this was homogenized, and squeegeed out at about 60° C. to a thickness of 1 mm on a polyethylene underlay.

After drying at 25° C. and a relative humidity of 30% over about three days, the film produced was peeled off from the PE underlay.

The fiber-reinforced gelatin film had a thickness of about 200 to 250 μm and a tear strength of about 22 N/mm² for an ultimate elongation of about 45%. A correspondingly produced, non-reinforced gelatin film had by contrast a tear strength of about 15 N/mm².

Production of the second layer was effected as described in Example 1, the box mold (without pericardial membrane) being filled with the foamed gelatin solution. A layer about 2 to 3 mm thick was cut from the dried gelatine sponge.

The fiber-reinforced gelatin film (first layer) and the gelatin sponge (second layer) were adhered to each other over their full surface area by means of a solution of bone gelatin (Bloom strength 160 g) and the composite material produced was then exposed to a second cross-linking, in the gas phase, with formaldehyde, as described in Example 1.

Instead of using a gelatin solution as adhesive, the bond between the two layers may alternatively be produced by the sponge, which has already been dried, being partially pressed into the squeegeed film while this is still not dry. In this manner, a stable bond over the full surface area may be achieved.

In a variant of this example, the cotton fibers were replaced by collagen fibers. Production of the films was effected as described above, save only that a suspension of 5 g of collagen fibers in 60 g of water or 10 g of collagen fibers in 90 g of water was added to the solution of gelatin and glycerin.

The dried films had a tear strength of about 25 N/mm² for an ultimate elongation of about 40% (5 g of fibers) and a tear strength of about 30 N/mm² for an ultimate elongation of about 27% (10 g of fibers), while the tear strength of a corresponding non-reinforced film was around about 17 N/mm².

The tear strengths of films reinforced with collagen fibers rose still further to about 28 N/mm² (5 g of fibers) and to about 33 N/mm² (10 g of fibers), by virtue of the second cross-linking in the gas phase.

### Example 3

### Population of a Composite Material According to the Invention with Chondrocvtes

This example describes the population of a composite material produced in accordance with Example 1, and cross-linked in two stages, with chondrocytes (cartilage cells) from pigs. This can be seen as a trial for transplantation of chondrocyte cells in which human cells, such as for example articular chondrocytes, are cultivated in vitro on the carrier material.

DMEM/10% FCS/Glutamine/Pen/Strep was used as culture medium, which is a standard medium for cultivation of mammalian cells. The composite material was washed with culture medium before it was populated. A million chondrocytes, suspended in 150 μm of culture medium, were then seeded-out onto the second layer of the composite material, per cm². The carrier material was then incubated in culture medium for four weeks at 37° C.

FIG. 2 shows an image, taken using an optical microscope, of the second layer of the composite material after incubation for two weeks. The cell nuclei 13 of the chondrocytes are distributed very uniformly over the entire volume. The sponge structure of the second layer had in the course of the two weeks broken down to a great extent and been replaced by the extracellular matrix 14 synthesized by the chondrocytes. The remainder of the sponge structure 15 is still to be seen, for example at the right hand edge of the illustration.

An this point, it should once again be mentioned that the breakdown of the material of the second layer takes place more quickly under these conditions than, as in the case of the experiment described in Example 1, in PBS buffer, which is inter alia to be attributed to enzymatic breakdown of the gelatin.

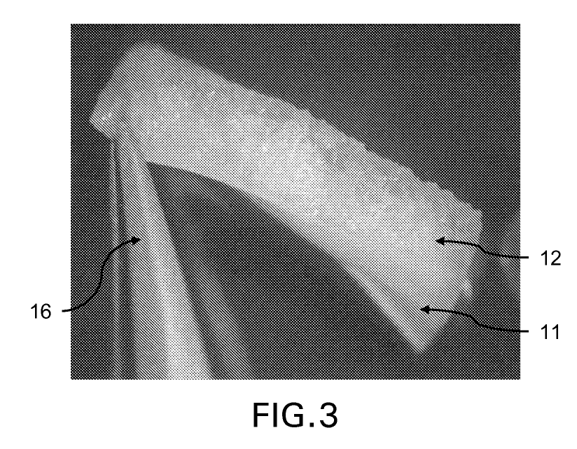

FIG. 3 shows a photographic illustration of the composite material according to the invention after a population time of four weeks. The composite material is held by a forceps 16, the second layer being oriented upwardly. Because of the extremely firm pericardial membrane 11, the composite material has, as previously a high degree of stability as to shape and can therefore be easily handled. In addition, there is also, after four weeks, a stable bond between the pericardial membrane 11 and the gelatin sponge 12 or the extracellular matrix formed in the sponge.

The results of this experiment show that corresponding tissue implants, which can be produced by making use of human chondrogenic cells, are highly suitable for use in the field of cell-based regeneration of cartilage.

1. A composite material, comprising
a first self-supporting layer, which comprises a first material which is insoluble, resorbable and non-gelling under physiological conditions; and
a second layer, comprising a cross-linked, gelatinous second material, the second layer having a mainly open-pored structure.

2. The composite material according to claim 1, the insoluble, resorbable and non-gelling first material being a planar material comprising collagen.

3. The composite material according to claim 2, the planar material being a natural membrane of animal origin.

4. The composite material according to claim 3, the membrane being a pericardial membrane.

5. The composite material according to claim 3, the membrane having a rough side which is oriented toward the second layer.

6. The composite material according to claim 1, the first material comprising a reinforcing material.

7. The composite material according to claim 6, the reinforcing material in the first layer having a fraction of 5% by weight or more.

8. The composite material according to claim 6, the reinforcing material in the first layer having a fraction of up to 60% by weight.

9. The composite material according to claim 6, the reinforcing material being selected from particulate and/or molecular reinforcing materials.

10. The composite material according to claim 9, the particulate reinforcing material comprising reinforcing fibers.

11. The composite material according to claim 10, the reinforcing fibers being selected from polysaccharide fibers and protein fibers, and from polyactide fibers and mixtures of any of the foregoing.

12. The composite material according to claim 9, the molecular reinforcing material being selected from polyactide polymers and their derivatives, cellulose derivatives, and chitosan and its derivatives.

13. The composite material according to claim 6, the first layer comprising a matrix in which the reinforcing material of the first material is embedded.

14. (canceled)

15. The composite material according to claim 13, the matrix comprising a cross-linked material containing gelatin.

16. The composite material according to claim 1, the first layer having a tear strength of 20 N/mm² or more.

17. (canceled)

18. The composite material according to claim 1, the second material being formed substantially entirely from gelatin.

19. The composite material according to claim 1, the gelatin having an endotoxin content, as determined by the LAL test, of 1,200 I.U./g or less.

20-23. (canceled)

24. The composite material according to claim 1, the second material having a content of excess cross-linking agent of about 0.2% by weight or less.

25-26. (canceled)

27. The composite material according to claim 1, the second layer having a fiber structure.

28. The composite material according to claim 27, the fiber structure being a textile, a knitted material, or a non-woven material.

29. The composite material according to claim 1, the second layer having a sponge structure.

30. The composite material according to claim 29, the sponge structure having an average pore diameter of 500 μm or less.

31. The composite material according to claim 30, the sponge structure having an average pore diameter of 100 to 300 μm.

32. The composite material according to claim 1, the second layer having a density from 10 to 100 g/l.

33-34. (canceled)

35. The composite material according to claim 1, the second layer being elastically deformable when in a hydrated state.

36. The composite material according to claim 35, the second layer decompressing to 90% or more within 10 minutes after having undergone a compression in volume by action of a pressure of 22 N/mm², in a hydrated state.

37. The composite material according to claim 1, the second layer, in a hydrated condition, having, after three days, a reduction in volume of less than 5% or an increase in volume.

38-40. (canceled)

41. The composite material according to claim 1, the first and second layers being bonded directly to one another.

42. The composite material according to claim 1, the first and second layers being bonded to one another by means of an adhesive.

43. The composite material according to claim 42, the adhesive comprising gelatin.

44. The composite material according to claim 1, the composite material having a thickness of 2 to 5 mm.

45. (canceled)

46. The composite material according to claim 1, further comprising a third layer bonded to the second layer.

47. The composite material according to claim 46, the third layer comprising a gelatinous material.

48. The composite material according to claim 47, the gelatinous material of the third layer being cross-linked.

49. The composite material according to claim 46, the third layer having a substantially closed structure.

50. The composite material according to claim 46, the third layer having a porous structure, the average pore diameter for the third layer being less than the average pore diameter of the structure of the second layer.

51. The composite material according to claim 46, the third layer comprising one or more calcium phosphates, apatites, or mixtures thereof.

52. A method for producing a composite material, comprising
providing a first self-supporting layer, which comprises a first material which is insoluble, resorbable and non-gelling under physiological conditions;
production of a second layer comprising a cross-linked, gelatinous second material, so that the second layer has a mainly open-pored structure; and
bonding the first and the second layer, the composite material being formed.

53. The method according to claim 52, the bonding between the first and the second layer being effected by an adhesive.

54. The method according to claim 52, the bonding between the first and the second layer being effected by partially pressing the second layer into the first layer, the first layer comprising a gelatinous matrix.

55. The method according to claim 52, the bonding between the first and the second layer being effected in the course of production of the second layer.

56. The method according to claim 55, comprising:
a) providing the first layer;
b) preparation of an aqueous solution of the gelatinous second material;
c) partial cross-linking of the second material in the solution;
d) foaming of the solution;
e) application of the foamed solution to the first layer; and
f) leaving the foamed solution to dry, the second layer being formed to have a mainly open-pored structure.

57. (canceled)

58. The method according to claim 56, further comprising:
g) further cross-linking the material comprised in the second layer.

59. The method according to claim 58, the cross-linking in g) being carried out by the action of a cross-linking agent in the gas phase.

60-61. (canceled)

62. The method according to claim 56, the cross-linking agent in c) being added to the solution in an amount of 600 to 5,500

63-64. (canceled)

65. The method according to claim 56, comprising removing excess cross-linking agent from the second layer after cross-linking.

66. The method according to claim 56, comprising subjecting the composite material to a thermal after-treatment at reduced pressure.

67. The method according to claim 66, the thermal after-treatment being carried out at a temperature of 80 to 160° C.

68. The method according to claim 52, further comprising application of a third layer to the second layer of the composite material.

69-78. (canceled)

79. An implant comprising a composite material, which comprises
a first self-supporting layer, which comprises a first material which is insoluble, resorbable and non-gelling under physiological conditions, and
a second layer, comprising a cross-linked, gelatinous second material, the second layer having a mainly open-pored structure
the implant further comprising cells which are embedded in the second layer of the composite material.

80. (canceled)

81. The implant according to claim 79, the cells being substantially uniformly distributed in the second layer of the composite material.

82-86. (canceled)

87. The composite material according to claim 19, the gelatin having an endotoxin content, as determined by the LAL test, of 200 I.U./g or less.

88. The implant according to claim 79, the cells being selected from chondrocytes, adult mesenchymalic stem cells, sinew cells, periosteum cells, and keratinocytes.

89. A method of treating a cartilage defect in a patient, comprising:
a) providing a composite material, comprising
a first self-supporting layer, which comprises a first material which is insoluble, resorbable and non-gelling under physiological conditions; and
a second layer, comprising a cross-linked, gelatinous second material, the second layer having a mainly open-pored structure
b) obtaining chondrocytes or stem cells of autologous or allogenic origin;
c) seeding-out the cells onto the second layer of the composite material; and
d) implanting the composite material at the location of the cartilage defect in the patient.

90. The method according to claim 89, the first layer of the composite material being oriented outwardly when implanting the composite material into the cartilage.

91. The method according to claim 89, further comprising cultivating the cells in vitro after seeding-out the cells and prior to implanting the composite material.
